(19) United States
(12) Plant Patent Application Publication
(10) Pub. No.: US 2013/0081172 P1
(43) Pub. Date: Mar. 28, 2013

(54) Phalaenopsis orchid plant named 'Honor'

(50) ;

(71) Applicant: Floricultura, Heemskerk (NL)

(72) Inventor: René Schoone, Assendelft (NL)

(73) Assignee: Floricultura, Heemskerk (NL)

(21) Appl. No.: 13/573,561

(22) Filed: Sep. 25, 2012

Related U.S. Application Data
(60) Provisional application No. 61/540,342, filed on Sep. 28, 2011.

(51) Int. Cl. A01H 5/00

(52) U.S. Cl. PLT/311

(57) ABSTRACT

A new and distinct Phalaenopsis plant named ‘Honor’ particularly characterized by flowers which are purple with darker purple marks and some white in the center; the labellum is purple with some yellow and white; plants which may be propagated economically and uniformly using tissue culture; plants which produce more than one inflorescence; long and sturdy inflorescences; and relatively short, dark-green foliage.

### CROSS-REFERENCE TO RELATED APPLICATIONS

This application claims priority to U.S. Provisional Application No. 61/540,342 filed Sep. 28, 2011. The disclosure of this prior application is hereby incorporated by reference.

### LATIN NAME OF THE GENUS AND SPECIES OF THE PLANT CLAIMED

Phalaenopsis hybrida

### VARIETY DENOMINATION

‘Honor’

### BACKGROUND OF THE INVENTION

The present invention relates to a new and distinct cultivar of Phalaenopsis plant, botanically known as Phalaenopsis of the Orchidaceae family, and hereinafter referred to by the cultivar name ‘Honor’.

Phalaenopsis comprises a genus of about 55 species of herbaceous perennials many of which, or the hybrids thereof, are suitable for cultivation in the home or greenhouse. Phalaenopsis is predominantly epiphytic or rock-dwelling, and is native to tropical Asia, the Malay Archipelago, and Oceania. The species typically has 2-ranked, fleshy, oblong or elliptic leaves affixed to a short central stem (monopodial growth), which vary in size from 5 to 8 inches to over 2 feet. The leaves may be entirely green or mottled with silver grey.

Phalaenopsis orchids, often referred to as ‘Moth Orchids’ in the horticultural trade, are frequently used to furnish cut flowers for the florist trade or sold as flowering potted-plants for home or interiorscape.

Phalaenopsis produces upright or pendent lateral racemes, often with many showy flowers which open in succession beginning with the lowermost. The flowers possess three sepals and three petals; the lateral ones being alike. The lowermost petal, called the labellum, is three-lobed and is often more brightly-colored than the other flower segments. Flower colors include various shades of pink, white, yellow and red-brown.

Phalaenopsis orchids are typically propagated from seeds. Asexual propagation of Phalaenopsis is often done from off-shoots which frequently arise from the lower bracts of the inflorescence. The resulting plants are detached from the mother plant and may be planted in a suitable substrate.

The new Phalaenopsis ‘Honor’ is a product of a controlled breeding program conducted by the inventor, Rene Schoone, in Strengweg, Heemskerk, The Netherlands. The objective of the breeding program was to develop a new Phalaenopsis cultivar particularly characterized by its attractive and unique colored flowers, economical propagation via tissue culture, rapid growth, and a plant dimension suitable for packaging and shipping to the market.

The new Phalaenopsis ‘Honor’ originated from a cross made by the inventor in 1999 in Strengweg, Heemskerk, The Netherlands. The female or seed parent is the Phalaenopsis cultivar designated ‘Chain Xen Diamond’, unpatented. The male or pollen parent is the Phalaenopsis cultivar designated ‘Happy Valentine’, unpatented. The new Phalaenopsis ‘Honor’ was discovered and selected by the inventor as a single flowering plant within the progeny of the stated cross in a controlled environment in 2006 in Strengweg, Heemskerk, The Netherlands.

Asexual reproduction of the new Phalaenopsis cultivar by tissue culture was first performed in July, 2007 in Cieweg 13, Heemskerk, The Netherlands, and has demonstrated that the combination of characteristics as herein disclosed for the new cultivar are firmly fixed and retained through successive generations of asexual reproduction. The new cultivar reproduces true to type.

### BRIEF DESCRIPTION OF THE INVENTION

The following traits have been repeatedly observed and are determined to be unique characteristics of ‘Honor’, which in combination distinguish this Phalaenopsis as a new and distinct cultivar:

1. flowers which are purple with darker purple marks and some white in the center; the labellum is purple with some yellow and white;

2. plant produces more than one inflorescence;

3. plants may be propagated economically and uniformly using tissue culture;

4. inflorescence is long and sturdy

5. relatively short, dark-green foliage

Presently, there is no commercial cultivar to which ‘Honor’ can be meaningfully compared.

### BRIEF DESCRIPTION OF THE PHOTOGRAPHS

The accompanying photographs illustrate the overall appearance of the new Phalaenopsis ‘Honor’ showing the colors as true as is reasonably possible with colored reproductions of this type. Colors in the photographs may differ slightly from the color values cited in the detailed botanical description, which accurately describe the color of ‘Honor’.

FIG. 1 shows a close-up view of the typical buds and flowers of ‘Honor’.

FIG. 2 shows a close-up view of the typical leaves of ‘Honor’

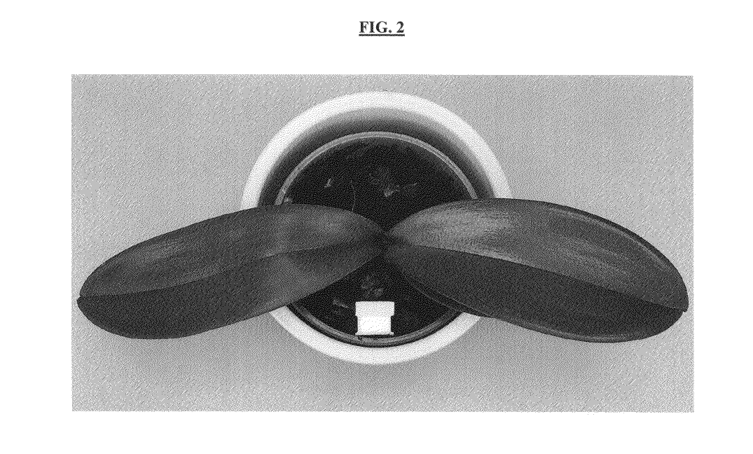

### DETAILED BOTANICAL DESCRIPTION

The new Phalaenopsis cultivar ‘Honor’ has not been observed under all possible environmental conditions. The phenotype of the new cultivar may vary with variations in environment such as temperature, light intensity, and day length without any change in the genotype of the plant.

The aforementioned photographs, together with the following observations, measurements and values describe plants of ‘Honor’ as grown in a greenhouse in Strengweg, Heemskerk, The Netherlands, under conditions which closely approximate those generally used in commercial practice. Initially, the ideal temperature to grow plants of ‘Honor’ is 27° C. during the day and at night. Then, during the flowering phase of ‘Honor’, the ideal growing temperature is 20-22° C. during the day and 18° C. at night. Light levels for growing ‘Honor’ are a minimum of 5,000 lux and a maximum of 10,000 lux. A balanced fertilizer with level of 200 ppm N, 87 ppm P, 168 ppm K is applied. Duration of growth of ‘Honor’ from potting size is between 10 and 14 months.

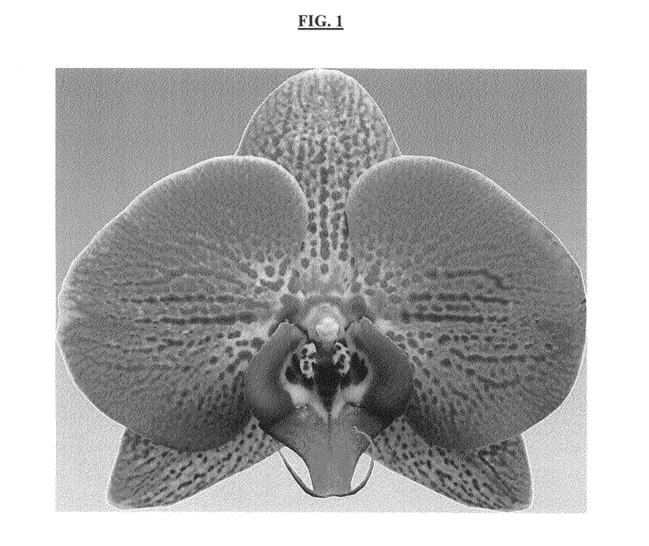

Color references are made to The Royal Horticultural Society Colour Chart (R.H.S.), 2007 edition, except where general colors of ordinary significance are used. Color values were taken under daylight conditions at approximately noon in Zaandammerweg, Assendelft, The Netherlands. The age of the ‘Honor’ plants described is 12 months after potting.
Classification:
   Botanical.—Phalaenopsis.
Parentage:
   Female or seed parent.—Phalaenopsis cultivar designated ‘Chain Xen Diamond’, unpatented.
   Male or pollen parent.—Phalaenopsis cultivar designated ‘Happy Valentine’, unpatented.
Propagation:
   Type.—tissue culture.
   Rooting habit and description.—Fleshy; approximately 4 mm-7 mm wide and green/yellow in color (RHS 146C); freely branching. It takes 12 weeks for plants growing in tissue culture to initiate roots.
Plant:
   Size at maturity.—Height: about 66 cm Spread: about 65 cm.
   Growth habit.—standard; dark-green leaves and relatively normal raceme.
   Vigor.—moderate.
   Crop time.—Following asexual propagation, at about 26 weeks 2 leaves appear; at about 30 weeks 3-4 leaves appear; after a cold treatment of about 4-8 weeks at a temperature of about 19° C. about 2 peduncles with flowers appear.
Foliage:
   Quantity per plant.—About 6 to 8 leaves are produced before flowering.
   Arrangement and attachment.—half up/horizontal and on two sides.
   Overall shape of leaf.—oval, tip is blunt and asymmetric.
   Texture.—smooth and leathery.
   Pubescence.—3 to 4 pairs of leaves.
   Mature leaf length.—about 20 cm.
   Mature leaf width.—about 7 to 10 cm.
   Mature leaf thickness.—about 2 mm.
   Mature leaf color (upper surface).—green (RHS N137A).
   Mature leaf color (under surface).—green (RHS N138A), edges are RHS 137A.
   Venation.—Pattern: parallel Color of midvein: upper surface RHS N137A, under surface RHS N138A.
Peduncle:
   Quantity per plant.—about 1 to 2.
   Number of flowers per peduncle.—about 6 to 8.
   Length.—about 72 cm.
   Diameter.—about 6 mm.
   Strength.—strong.
   Aspect.—upright.
   Texture.—glabrous and smooth.
   Color.—yellow/green RHS 147A.
   Internode.—Length: about 37 to 50 mm.
Inflorescence description:
   Appearance.—upright to slightly pendant, racemose inflorescence with bilaterally symmetrical flowers that open in succession beginning with the lowermost flower.
   Inflorescence size.—Height (from base to tip): about 18 to 24 mm Diameter (at midpoint): about 15 to 23 mm Shape: egg-shaped Color: Main color is green/yellow (RHS 145A), edges and extension are purple (RHS N79C).
   Flowering time.—For an untreated plant (flowering plant that has not undergone cold-treatment where the plant grows at a temperature of 18° C. to 19° C. for about 4 to 8 weeks after a period of about 30 weeks at a temperature of 25° C.), 2 racemes appear with about 16 to 20 flower buds and flowers per inflorescence. First flowers can be expected approximately 4 to 6 months after planting a plant with a leaf diameter of 3 to 5 cm. Flowers persistent.
   Flowering longevity.—On the plant: about 4 to 6 months; lastingness of cut flowers: has not been observed.
   Fragrance.—no fragrance.
   Flower.—Rate of opening: Flowers fully opened about 2 to 3 days after tepal separation. Orientation at opening: slanted upward and outward. Shape: see FIG. 1 Size: Height: about 74 mm Diameter: about 87 mm Depth of tube: about 9 mm.
   Tepals.—Quantity and arrangement: six tepals that are trimerous, overlapping and arranged in 2 whorls. Petals are more pronounced than sepals.
   Petals.—Arrangement: Inner whorl of tepals comprises 3 petals, 2 lateral petals and labellum. 2 lateral petals: Overall shape: broadly ovate and weakly cupped Apex: oval Margin: weakly undulate Length: about 50 to 60 mm Width: about 40 mm Texture: Upper surface: smooth and satiny Under surface: smooth and satiny Color (when fully opened): upper surface: Main color is purple/violet (RHS N80A) with purple strips/spots (RHS N79B) and some white at the base (RHS NN155D). Under surface: White (RHS NN155D and purple (RHS 77B) Labellum: Overall shape: 3-lobed with 2 prominent callosities at central junction of the lateral lobes and base of the midlobe. Lateral lobes of labellum fold upward about the column; the midlobe extends forward and is terminated by 2 short filiform appendages at the apex. Lateral lobes of the labellum are ovate in shape while the midlobe is triangular with a bump and a rib on it Margin: entire and weakly undulate Length: about 25 mm Width (not flattened): about 25 mm Texture: Upper surface: smooth and satiny Color (when fully opened): Main color is purple (RHS N78A). At the base some yellow (RHS 7A) and some white (RHS NN155D). At the lateral lobs are some purple marks (RHS N79B). Chirri is purple (RHS N78A). the tips of the chirri are yellow (RHS 7A). Underside: Main color is purple (RHS N78A). At the base some white (RHS NN155D), some yellow (RHS 7A) and some purple (RHS N78C). Chirri: long Pestle Length: About 7 mm Width (not flattened): about 6 mm Color: White (RHS NN155D), some yellow spots (RHS 7A) and on the sides and on top purple (RHS N77A).
   Sepals.—Arrangement: Outer whorl of tepals comprises 3 sepals. Overall shape: elliptical and weakly cupped Length: about 35 to 45 mm Width: about 27 to 34 mm Texture: Upper & under surface: smooth and satiny Color (when fully opened): Dorsal sepal: Main color is purple (RHS N80A) At the base purple (RHS N80B). Stripes/spots are purple (RHS N79B). Under side: Base and center are purple (RHS 153A) and the edges (RHS N79D). Lateral sepals: Main color is purple (RHS 80B) with purple stripes/spots (RHS N79B). At the base some white (RHS NN155C) and yellow/green (RHS 147C). Under side: main color is purple (RHS 80B & N80A), at the base some yellow/green (RHS 145C), the veins are purple (RHS N79C)
Reproductive organs:
   Arrangemen.—The stamens, style and stigmas are fused into a single, short structure called the column, possessing one terminal anther with pollen grains united into a pollinia, which are covered by an anther cap. The stigma is located under the column behind the pollinia. The ovary is inferior with three carpels present. The plant has not produced seed.
   Column.—Length: about 9 mm Diameter: about 7 mm Color: purple (RHS N78A).
   Pollinia.—Quantity: Two Size: about 1 mm Color: orange (RHS 24A).
   Ovary.—Length: about 4 mm Diameter: about 7 mm Color: white (RHS N155C) with purple (RHS N79C).
   Pedicel.—Length: about 37 to 50 mm Diameter: about 4 mm Texture: glabrous and smooth Color: at the base yellow/green (RHS 144A) with extension to white (RHS N155C) and purple (RHS N80B).
Disease/pest resistance/susceptibility: No specific resistance or susceptibility observed
Temperature tolerance: Tolerant to a low temperature of about 15° C. and to a high temperature about 30° C.

What is claimed is:

1. A new and distinct Phalaenopsis plant named ‘Honor’, as illustrated and described herein.
